(19) United States
(12) Patent Application Publication
(10) Pub. No.: US 2010/0068340 A1
(43) Pub. Date: Mar. 18, 2010

(54) ICE-STRUCTURING PEPTIDES OF LACTIC ORIGIN

(75) Inventors: Hans-Juergen Erich Wille, St. Martin Le Noeud (FR); Joselio Batista Vieira, York (GB); Cornelis Gijsbertus De Kruif, KK Woudenberg (NL); Theodorus Arnoldus Gerardus Floris, AT Arnhem (NL); Karel Joseph Slangen, DW Ede (NL)

Correspondence Address: K&L Gates LLP, P.O. Box 1135, CHICAGO, IL, 60690, US

(73) Assignee: NESTEC S.A., Vevey (CH)

(21) Appl. No.: 12/443,084

(22) PCT Filed: Sep. 19, 2007
(86) PCT No.: PCT/EP07/59864

§ 371 Date: Apr. 14, 2009

(30) Foreign Application Priority Data
Oct. 20, 2006 (EP) 06022065.4

(51) Int. Cl. A23G 9/40; C07K 2/00; A23G 9/52; A23G 9/48; A23G 9/46; A23G 9/36

(52) U.S. Cl. 426/42; 530/300; 426/334; 426/66; 426/100; 426/329

(57) ABSTRACT

The present invention relates to the use of peptides of lactic origin as ice-structuring agents, to their use in the manufacture of frozen products, and to the frozen products comprising them.

### FIELD OF THE INVENTION

The present invention relates to the use of peptides of lactic origin as ice-structuring agents, to their use in the manufacture of frozen products, and to the frozen products comprising them.

### BACKGROUND OF THE INVENTION

Ice-structuring proteins (ISPs) or abusively named anti-freeze proteins (AFPs) naturally occur in a range of species that are susceptible to freeze damage, i.e. to species that are found in sub-zero environments. They have evolved in nature to help many different organisms e.g. fish, insects, plants and bacteria, to survive in these cold environments. To date, fish from cold climates has been considered as the main source of ISPs (cf. for example WO 9702343 (Unilever), EP 0788745 (Nestlé)). Plant sources of ISPs are also described in for instance EP 0959689 (Unilever), EP 0918863 B1 (Unilever), EP 1049713 (Unilever), EP 1049783 B1 (Unilever), EP 1276763 (Unilever). Further, EP 1240188 (Unilever) discloses ISPs isolated from bacterial sources from low-temperature environments. WO 9804147 (Unilever) reports the isolation of peptides that inhibit ice-crystal growth, derived from plants such as rye or grass.

It is thought that ISPs achieve their function by binding to specific planes of the ice crystals and by minimising recrystallisation (Biophysical Journal, February 1991, 409-418). Inhibition of ice recrystallisation, also referred to as ice crystal growth suppression (Cryobiology, 25, 55-60, 1988) is a property of ISPs that can be tested by comparing at a certain point in time the ice crystals in the presence of ISP and in the absence of ISP. The application of this method in the testing of fish ISPs is described in U.S. Pat. No. 5,118,792 (DNA Plant technology Corporation). Thus, it has been reported that ISPs from Antarctic fish are very effective in minimizing ice crystal growth (Cryobiology 32:23-34, 1995). Compared to fish ISPs, an ISP found recently in rye grass (Lolium perenne) is reported to be even 200 times more effective (in molar terms) in inhibiting recrystallisation (Nature, 406(6793):256, 2000).

Another property of ISPs is their ability to influence the shape of ice crystals. This property stems from selective binding of ISPs to certain faces of the ice crystals and therewith limiting crystal growth in certain directions. The presence of ice crystals having a hexagonal bipyramid shape is then considered indicative of the presence of ISP. This method is described for testing the activity of extracellular winter rye ISPs in WO 92/22581 (University of Waterloo).

ISPs also have the ability to inhibit the activity of ice nucleating substances. This interaction between an ISP and ice nucleator may for example result in increasing thermal hysteresis (WO 96/40973—University of Notre Dame du Lac). Thermal hysteresis is characterised by a lowering of the apparent freezing temperature of a solution without affecting the melting temperature. Thus, the identification of sources of ISPs by thermal hysteresis tests is widely described in the literature (e.g. John G. Duman, Cryobiology, 30, 322-328, 1993).

It has been suggested that it is the tertiary structure of these proteins which allows them to interact with ice (cf. Fletcher et al., Annu. Rev. Physiol., 2001, 63:359-390). Thus, all these properties render ISPs applicable to a range of potential uses. Most importantly, ISPs have been suggested for improving the freezing tolerance of products. Frozen products can be subjected to temperature fluctuations leading to an increase in ice crystal size and thus to textural defects. ISPs thus enable frozen products to withstand temperature fluctuations that may occur during packaging, storing, manufacturing etc., thus allowing them to keep a desirable texture.

The use of ISPs in the field of frozen foods has been widely reported in WO2006042632 (Unilever), EP 1541034 (Unilever), EP 1158866 (Unilever), U.S. Pat. No. 6,914,043 (Unilever), EP 0966206 (Unilever), WO 9841107 (Unilever), WO 9841109 (Unilever), EP 1049383 (Unilever); EP 1158865 (Unilever), EP 1158863 (Unilever), EP 1158862 (Unilever), EP 1158864 (Unilever), WO 98/04146 (Unilever), EP 1417892 (Unilever), WO 03055320 (Unilever), US 20040048962 (Unilever).

However, sources of ISPs have been limited to sources from sub-zero environments and/or to the use of genetically modified organisms (GMOs) for producing these proteins. For instance, WO 9403617 (Unilever) discloses the production of ISPs from yeast and their possible use in ice cream. WO 9611586 (HSC R&D Ltd—Seabright Corporation Ltd) describes fish ISPs produced by microbes. Others ISPs have mainly been obtained by enzymatic and chemical modification. For example, WO 9013571 (DNA Plant technology Corporation) discloses ISP peptides produced chemically or by recombinant DNA techniques from plants.

These techniques are subject to much controversy and the resulting “GMO labelled” products are not always appealing to the consumer.

### OBJECT OF THE INVENTION

It is thus an object of the invention to provide an alternative source of ice-structuring agent which can be used in frozen products and which avoids the need to use genetically manipulated additives.

### SUMMARY OF THE INVENTION

Accordingly, this object is solved by the features of the independent claims. The dependent claims further develop the central idea of the invention.

Thus, in a first aspect, the invention provides a frozen food product comprising at least one ice-structuring peptide derived from milk protein.

In a second aspect, the invention relates to the use of a peptide derived from milk protein as ice-structuring agent.

A process for improving the heat shock resistance of frozen confectionery product comprising the steps of:
a. Cleaving a milk protein into peptides
b. Isolating the peptides obtained in the previous step and
c. Using said peptides in the manufacture of a frozen confectionery product
   also forms part of the invention.

Finally, the present invention also encompasses an ice-structuring peptide obtainable by enzymatic cleavage of casein.

### FIGURES

The present invention is described hereinafter with reference to some of its embodiments (or reference embodiment) shown in the figures, wherein

FIG. 1 shows the evolution of ice crystal size with increasing heat shock periods (before heat shock, after 2 weeks heat shock and after 3 weeks heat shock) for a standard ice cream mix and for an ice cream comprising Peptigen IF-2050, a commercial casein hydrolysate (Aria Food Ingredient—Denmark).

FIG. 2 shows the evolution of ice crystal size with increasing heat shock periods (before heat shock, after 2 weeks heat shock and after 3 weeks heat shock) for a standard ice cream mix, for an ice cream comprising Peptigen IF-2050, for an ice cream comprising peptides obtained from hydrolysis of casein with papain and for an ice cream comprising peptides obtained from hydrolysis of casein with trypsin.

FIGS. 3a and 3b are pictures of ice crystals after 3 weeks heat shock in ice cream for a standard mix (FIG. 3a) and for an ice cream comprising Peptigen IF-2050 (FIG. 3b), wherein the scale bar represents 100 microns.

### DETAILED DESCRIPTION OF THE INVENTION

The present invention relates to frozen food products which comprise at least one ice-structuring peptide. By “peptide” is meant a chain of up to 50 amino acids linked by peptide bonds. The peptides of the invention are not proteins and do not comprise a tertiary structure. They are protein hydrolysates. The weight of said peptide is less than 1 kDa. By “ice-structuring” is meant that the peptide is able to interact at the ice crystal interface, in particular to inhibit ice crystal growth. This function is in contrast to and distinguishable from the aerating/air cell stabilising function of some peptides which occurs at the air cell interfaces.

The peptides used in the present invention are derived from milk protein. Milk proteins include casein and whey protein. Preferably, the milk protein used is casein. By “casein” is meant casein as it is found naturally, i.e. casein which has not been modified chemically. According to the invention, this definition includes casein as such and water-soluble caseinates, for example alkali metal, alkaline earth metal and ammonium caseinates.

The peptide may be derived by enzymatic or chemical cleavage of said milk protein. In a preferred embodiment, the milk protein is treated with an enzyme, which may be selected from any protease enzyme capable of hydrolysing the milk protein into peptides. More preferably, the enzyme is selected from trypsin, papain, neutrase or mixtures thereof. According to the invention, upon treatment of milk protein with an enzyme, the protein is cleaved into peptides which may be used as ice-structuring agents. These are capable of controlling frozen food stability by inhibiting crystal growth, thus improving the quality of the product.

Alternatively, it has been found that commercially available milk protein hydrolysates may be used in the present invention. It is thus thought that said commercial hydrolysates may comprise peptides according to the invention, i.e. peptides which may be used as ice-structuring agents. Such hydrolysates are for example sold under the name of Peptigen, Peptone, Peptopro etc.

Crystal growth can be measured by crystal size analysis using a computer-controlled image analyser. The size of crystals is usually measured by the diameter distribution over the volume (i.e. over the amount of ice crystals evaluated). Thus a Dv(0.50) represents the value of the maximum diameter of 50% of the total number of ice crystals evaluated. Dv(0.90) represents the value of the maximum diameter of 90% of the total number of ice crystals evaluated. By using the peptides of the invention, the crystal size of a sugar solution at −11° C. containing said peptides is reduced by 15%-25% compared to a reference sugar solution at −11° C. with no peptides (cf. Table 2).

The effect of the peptides is further evidenced by heat shock treatment. By “heat shock” is meant the inevitable temperature cycling during storage and distribution that creates ice crystals growth and other deterioration due to structural change. This “heat shock” is reproduced by a process, which is a defined cycle of thermal changes inflicted on the product. The product is placed inside a cabinet set at −20° C., which is automatically switched on for 19 hours and then switched off for 5 hours using a 24-hour timer in order to provoke a thermal shock.

These analyses led to the surprising conclusion that certain casein-derived peptides have a visible and measurable action on the inhibition of ice-crystal growth.

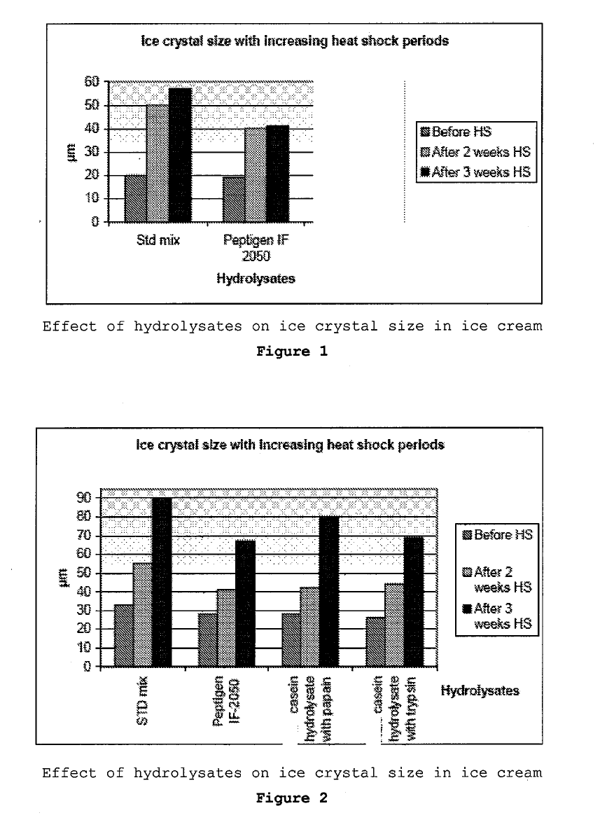

Indeed, referring to FIGS. 1, 2 and 3a and 3b, it can be seen that although the crystal size in ice cream grows, in particular after 2 weeks of heat shock, by using the ice-structuring peptides according to the invention a smaller crystal size is obtained.

The manufacturing conditions of some of said peptides which provide anti-freeze activity are given in table 1.

Thus, the present invention relates to frozen food product of the invention which comprise the peptide of the invention in an amount between 0.0001-10%, preferably in an amount between 0.001-5%, more preferably in an amount between 0.01-1% by weight of the composition.

The frozen food product of the invention may be any food product. Preferably, it is a food confectionery product which may be ice cream, water ice, sorbet, frozen yogurt, mellorine etc. The product may comprise inclusions in the form of chocolate pieces, nuts, pieces of fruits etc. The product may also comprise a coating, such as e.g. a chocolate coating or a fruit coating etc. The coating itself may also contain inclusions. The frozen food product may be aerated or non-aerated. Aerated frozen confections preferably have an overrun of from 30% to 200%, more preferably from 50% to 150%. For example, the level of overrun in ice cream is typically from about 70% to 100%, and in confectionery such as mousses the overrun can be as high as 200 to 250 wt %, whereas the overrun in milk ices is from 25 to 35%.

Thus, the invention encompasses the use of a peptide derived from milk protein as ice-structuring agent. The milk protein is preferably casein and the peptide may be derived from the milk protein by chemical or enzymatic cleavage of the protein.

According to another embodiment of the invention, the peptide may be obtained through fractionation of the substrate obtained by chemical or enzymatic cleavage of the protein. This provides the advantage of enriching the active principle and thus to work at lower concentrations.

According to the invention, the ice-structuring peptide is preferably used in frozen confectionery products.

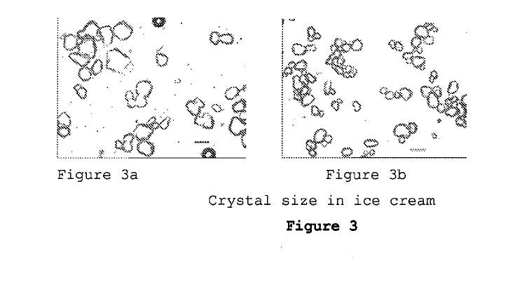

Referring to FIG. 3b, it can be seen that by using the peptides according to the invention, the ice crystals are smaller compared to a standard ice cream mix (FIG. 3a).

Furthermore, it has been observed that the shape of ice crystals in ice cream maintains an essentially round aspect. This effect is surprising in view of the fact that regular anti-freeze proteins used in the art tend to modify the structure of ice crystals. Indeed, ice crystals found in frozen products containing ISPs tend to have an elongated, rectangular shape which affects the texture of the product by increasing its hardness. By the present invention, a frozen product having a smooth, soft texture may be obtained while still being resistant to temperature fluctuations.

In a further aspect, the invention thus provides a method for improving the heat shock resistance of frozen confectionery products.

The first step in the method consists in the cleavage of the milk protein. Preferably the milk protein is casein. Cleavage may be carried out chemically or enzymatically. A preferred process is enzymatic hydrolysis of the milk protein in order to yield peptides. For the hydrolysis of milk protein, the enzyme/substrate ratio is from 1/100 to 1/500, and preferably 1/250 w/w.

The peptides obtained may vary widely depending on the conditions used e.g. incubation temperature, incubation time, the pH of the solution etc. According to the invention, it has been found that an incubation temperature between 45° C.-70° C., an incubation time between 5 and 480 minutes and a pH of the solution between 6.5 and 8.5 are preferred conditions in order to obtain different peptides which are all efficient ice-structuring compounds.

The enzyme used may be selected from any protease enzyme. Preferably, it is selected from trypsin, papain, neutrase or mixtures thereof.

Thus, the milk protein is incubated with the desired enzyme under determined conditions. After the desired period of time, the enzyme is then inactivated and the substrate is collected. Peptides are then isolated from the substrate and may be used directly in the production of a frozen product. Alternatively, the substrate may further be subjected to fractionation, after which the peptides are isolated and used as ice-structuring agents in the manufacture of a frozen product. The peptides may also be further purified prior to use. The frozen product may be manufactured by any method known to the skilled person. Further, the peptides of the invention may be added at any stage during manufacture of the frozen product, more preferably during mix preparation, before maturation time.

The invention thus also relates to ice-structuring peptides obtainable by enzymatic cleavage of casein. The enzyme cleavage may be carried out by any embodiment of a process described above.

In summary, the present invention provides a way to produce frozen products, and in particular frozen confectionery products which are smooth and stable after heat shock. It also provides for natural frozen products which have a “clean” label and are free of GMO additives. Furthermore, the modification of the ice crystal structure observed when using known ISPs is no longer observed. This yields a product which retains essentially circular ice crystals and maintains a smooth, palatable texture after heat shock.

The present invention is further illustrated by the following non-limiting examples.

### Examples

### Example 1

### Manufacture of Peptides

Commercial sodium caseinates were subjected to the action of different enzymes and the resulting hydrolysates were tested for their inhibitory activity on ice-crystal growth.

The hydrolysis is carried out according to the following procedure. The sodium caseinate protein isolate is dissolved at a concentration of 5% of proteins in water. The pH is adjusted to the desired pH according to table 1 by adding either sodium hydroxide, 1N NaOH, or hydrochloric acid, 1N HCl. The substrate is then brought to the desired temperature. Finally, the enzyme is added. During reaction, the pH is not adjusted.

For the hydrolysis of the caseinate, the enzyme/substrate ratio is from 1/100 to 1/500, and preferably 1/250 weight for weight.

Depending on the reaction conditions, the reaction time can go from 5 min up to 1200 min.

### Example 2

### Sample Preparation for Analysis

50 μl of the reaction product (obtained by the method described in example 1) are mixed with RP-HPLC buffer for chromatographic analyses. Simultaneously, 1.5 ml of the reaction product are heated at 90° C. for 15 minutes with the aim of inactivating the enzymes. After heating, the sample is centrifuged for 15 minutes at 14 000 revolutions/min (rpm), in order to eliminate any possible precipitates. The supernatant is lyophilized for the analysis of ice-crystal recrystallization.

### Example 3

### HPLC Analysis

The reverse-phase HPLC analysis is carried out on the samples obtained in example 2 according to the method described in S. Visser, C. J. Stangen and H. S. Rollema (1991) “Phenotyping of bovine milk proteins by reversed-phase high-performance liquid chromatography”, J. Chromatography, 548, pp. 361-370. The separation is based mainly on the differences in hydrophobicity of the proteins and of the peptides. The detection is carried out by UV absorption at 22 nm.

### Example 4

### Ice Recrystallisation Test

To evaluate the effect of the lactic protein hydrolysates on ice-crystal recrystallization, an analysis of ice-crystal recrystallization is carried out.

Materials:
Cryomicroscope consisting of:
(1) Provis AX 70 Olympus microscope
(2) Linkam BCS 196 cold stage
(3) Linkam Nitrogen Pump LNP 93/2 fitted with 2L Dewar
(4) Linkam Temperature Controller TP93
(5) 3CCD Colour Video Camera (DXC-950P Sony)
14 mm Round Cover Glass No 1 Deckglaser
10 μL Microsyringe #701 Hamilton
Quartz Coverslips Holder Linkam THMS/Q
Analytical Balance AT400 Mettler Toledo
250 ml Erlenmeyer
20 ml glass vials
Liquid Nitrogen Air Liquide
Distilled water
D(+)-Saccharose #16104 Sigma-Aldrich

The lyophilized hydrolysate obtained according to the procedure described above is dissolved in a 40% solution of sucrose in water. The final solution contains 5% by weight of lyophilized hydrolysate. A 40% solution of sucrose in water is used as a reference sample. A solution of peptides that inhibit ice-crystal growth (ISP type 1) in a 40% solution of sucrose in water is used as a positive control. The samples are analysed by observation under a microscope of Polyvar type sold by Reichert-Jung, Harnalser Hauptstrasse 219, Vienna, Austria, equipped with a Linkham temperature regulator sold by Linkham Scientific Instruments Ltd, Tadworth UK. The temperature regulator is pre-calibrated with n-dodecane (melting point: −9.6° C.) and n-decane (melting point: −29.7° C.)

A 2 μl sample is placed on a quartz cell covered with a circular cap. The quartz cell is placed on the temperature regulator and then cooled to −100° C. at a rate of 90° C. per minute. At −100° C., the sample is left to equilibrate for 2 minutes, and then reheated to −11° C. at a rate of 30° C. per minute. The time zero of the analysis is taken at the instant the sample reaches −11° C. During the first two minutes of the analysis, the microscope is regulated in order to ensure a good image and sufficient crystals for a significant analysis. After 2 minutes, the images from the microscope are acquired and stored using a video recorder software with a pre-defined time lapse (2 minutes). The images are recorded for each hydrolysate for 2 h at constant temperature. Results are shown in table 2.

For each sample taken, the 40% sucrose solution is taken as a reference. For this solution, the ice crystals reach the average maximum size that can be reached for a given cooling/heating cycle since no ice-crystal growth inhibitor is present. The results obtained for the various hydrolysates can thus be compared with the microscope images of the ice crystals in the reference sucrose sample. By comparing the microscope images of the state of the crystals after one hour at −11° C. with the image obtained for the reference sucrose solution, it is possible to establish whether or not an ice-crystal growth inhibition effect is observed for each hydrolysate tested.

Micrographs show the evolution of ice crystal during a typical experiment using the conditions described herein.

TABLE 1
Hydrolysates with ice-structuring activity

| | | Incubation | | Temperature |
|---|---|---|---|---|
| Protein | Enzyme | time | pH | ° C. |
| Casein | Papain | 480 min | 7 | 70 |
| Casein | Trypsin | 480 min | 8 | 45 |
| Casein | Neutrase | 5 min | 7 | 45 |
| Casein | Papain | 5 min | 7 | 70 |
| Casein | Trypsin | 5 min | 8 | 45 |

All these hydrolysates were the subject of a crystal size analysis using a computer-controlled image analyser. The results are reported in table 2.

TABLE 2
Crystal size analysis

| | Crystal size (μm) | | Crystal size (μm) | |
|---|---|---|---|---|
| | after 1 h at −11° C. | | after 2 h at −11° C. | |
| | Dv(0.50) | Dv(0.90) | Dv(0.50) | Dv(0.90) |
| 40% sucrose | 16.0 | 24.5 | 18.7 | 30 |
| solution | | | | |
| (reference) | | | | |
| Hydrol. | 12.6 | 18.0 | 16.7 | 26.6 |
| Caseinate with | | | | |
| Papain 480 min | | | | |
| Hydrol. | 13.3 | 19.0 | 16.8 | 25 |
| Caseinate with | | | | |
| Trypsin 480 min | | | | |
| Hydrol. | 13.0 | 19.1 | 15.4 | 22 |
| Caseinate with | | | | |
| Neutrase 5 min | | | | |
| Hydrol. | 12.7 | 18.6 | 16.5 | 22.5 |
| Caseinate with | | | | |
| Papain 5 min | | | | |
| Hydrol. | 12.3 | 17.5 | 15 | 22.1 |
| Caseinate with | | | | |
| Trypsin 5 min | | | | |

Dv (0.50) represents the value of the maximum diameter of 50% of the total number of ice crystals evaluated;
Dv (0.90) represents the value of the maximum diameter of 90% of the total number of ice crystals evaluated.

The results of this analysis confirm that the size of the ice crystals obtained is smaller in the five solutions containing the casein hydrolysates selected than in the 40% sucrose reference solution without agent for inhibiting ice-crystal growth. This shows the inhibitory effect on ice-crystal growth of these five hydrolysates.

### Example 3

### Ice Cream Recipes Used for Trials

The following recipes were used in the trials at 1, 5 and 10% in ice cream. The standard mixes with an equivalent total solids (TS) content were used as reference.

| | Std mix | | Std mix | | Std mix | |
|---|---|---|---|---|---|---|
| Ingredients | for 1% | 1% P | for 5% | 5% P | for 10% | 10% P |
| Water | 61.5 | 61.5 | 61.5 | 61.5 | 61.5 | 61.5 |
| Skimmed milk | 2 | 2 | 2 | 2 | 2 | 2 |
| powder | | | | | | |
| Sweet whey | 8 | 8 | 8 | 8 | 6.5 | 6.5 |
| powder | | | | | | |
| Sugar | 13 | 13 | 13 | 13 | 11 | 11 |
| Glucose syrup | 1 | 1 | 1 | 1 | 1 | 1 |
| DE40 | | | | | | |
| Coconut fat | 9 | 9 | 9 | 9 | 7.5 | 7.5 |
| Emulsifier | 0.3 | 0.3 | 0.3 | 0.3 | 0.3 | 0.3 |
| Guar gum | 0.2 | 0.2 | 0.2 | 0.2 | 0.2 | 0.2 |
| Glucose syrup | 5 | 4 | 5 | | 10 | |
| DE 20-23 | | | | | | |
| Peptides | | 1 | | 5 | | 10 |

Std: standard
P: peptides of the invention
TS: 38%

The freezing was performed after 24 hours aging on a KF 80 (Hoyer) using the following parameters:

| | | |
|---|---|---|
| Dasher speed | 860 | rpm |
| Flow rate | 70 | l/h |
| Barrel relative pressure | 3 | bar |
| Freezing temperature | −5.7° | C. |

| | |
|---|---|
| Overrun | 100% |

Technical tasting carried out after 3 weeks of heat shock found that the standard mix was very crystallised in the mouth. The perception of ice crystals after heat shock was lower in ice cream samples containing the peptides of the invention.

1. Frozen food product comprising at least one ice-structuring peptide that is derived from a milk protein.

2. Frozen food product according to claim 1, wherein the peptide is derived by a cleavage of the milk protein using a process selected from the group consisting of enzymatic and chemical.

3. Frozen food product according to claim 1, wherein the milk protein is casein.

4. Frozen food product according to claim 1, wherein the peptide is present in an amount of between 0.0001-10% by weight of the composition.

5. Frozen food product according to claim 1, wherein the peptide is present in an amount of between 0.001-5% by weight of the composition.

6. Frozen food product according to claim 1, wherein the peptide is present in an amount of between 0.01-1% by weight of the composition.

7. Frozen food product according to claim 1, which is selected from the group consisting of ice cream, water ice, sorbet, frozen yogurt, and mellorine.

8. Frozen food product according to claim 1, comprising inclusions and coatings.

9. Frozen food product according to claim 1, which is aerated.

10. A process for making a food product comprising using a peptide derived from a milk protein as an ice-structuring agent.

11. The process according to claim 10, wherein the milk protein is casein.

12. The process according to claim 10, wherein the peptide is derived by cleaving the milk protein using a process selected from the group consisting of chemical and enzymatic.

13. The process according to claim 10, wherein the peptide is obtained through fractionation of a chemical or enzymatic cleavage substrate.

14. The process according to claim 10 comprising the step of producing a frozen confectionery product.

15. Process for improving the heat shock resistance of a frozen confectionery product comprising the steps of:
cleaving a milk protein into peptides;
isolating the peptides so obtained;
using the peptides to manufacture a frozen confectionery product.

16. Process of claim 15, comprising performing a fractionation step on the peptides.

17. Process according to claim 15, wherein the milk protein is casein.

18. Process according to claim 15, wherein the cleavage step is selected from the group consisting of enzymatic and chemical.

19. Process according to claim 18, wherein the enzymatic cleavage is carried out with an enzyme selected from the group consisting of trypsin, papain, neutrase and mixtures thereof.

20. Process according to claim 18, wherein the enzymatic cleavage is performed for a period of 5 to 480 min.

21. Process according to claim 18, wherein the enzymatic cleavage is performed at a temperature between 45° C. and 70° C.

22. Process according to claim 18, wherein the enzymatic cleavage is performed at a pH between 6.5 and 8.5.

23. Ice-structuring peptide obtainable by an enzymatic cleavage of casein.

24. Peptide according to claim 23, wherein the cleavage of casein is performed using an enzyme selected from the group consisting of trypsin, papain, neutrase and mixtures thereof.

25. Peptide according to claim 23, wherein the cleavage is performed for 5 to 480 minutes, at a temperature between 45° C.-70° C. and a pH between 6.5 and 8.5.
